(19) United States
(12) Patent Application Publication
(10) Pub. No.: US 2014/0377353 A1
(43) Pub. Date: Dec. 25, 2014

(54) PHARMACEUTICAL PRODUCT TO INDUCE ENDOGENOUS EXACERBATED RELEASE OF GUT HORMONES PYY AND GLP1 WITH THERAPEUTIC EFFECT ON OBESITY AND TYPE 2 DIABETES MELLITUS

(71) Applicant: GILBERTO BORGES DE BRITO, SAO JOSE DO RIO PRETO (BR)

(72) Inventor: GILBERTO BORGES DE BRITO, SAO JOSE DO RIO PRETO (BR)

(21) Appl. No.: 13/924,984

(22) Filed: Jun. 24, 2013

(51) Int. Cl. A61K 9/50; A61K 9/28; A61K 31/575; A61K 31/702; A61K 31/715; A61K 31/20; A61K 38/16; A61K 31/23

(52) U.S. Cl. CPC A61K 9/5021; A61K 38/16; A61K 9/2833; A61K 31/23; A61K 31/702; A61K 31/715; A61K 31/20; A61K 31/575
USPC 424/482; 514/4.9; 424/490; 424/497; 424/474

(57) ABSTRACT

The present invention concerns a pharmaceutical product to induce endogenous exacerbated release of gut hormones, pyy and glp1, with therapeutic effect on obesity and type 2 diabetes mellitus. In tablets form, suitable dosage forms such as pills capable of disintegration from a certain range of Ph or specific Ph range between 6.0 and 7.0, so that the undigested or partially hydrolyzed content is released in the distal jejunum and preferably in the ileum/proximal colon. And by contact with macro-nutrient molecules, or their hydrolyzed fragments, such cells suffer intense amplified stimulus and secrete the said hormones, released at rates that are therapeutics for obesity of any degree and also for type II diabetes; mimicking that produced and performed in bariatric and metabolic surgeries by a noninvasive method.

Refers this descriptive report to a patent application of invention of a pharmaceutical product to induce endogenous exacerbated/stimulated/amplified release of gut hormones, particularly and especially PYY (peptide YY) and GLP-1 (glucagon-like polypeptide 1) by means of release/delivery of a mixture of non-digested or partially digested macro-nutrients, with or without micronutrients and/or bile salts, in any possible combination, which show effective for this purpose, directly into the ileum and/or proximal colon.

This mixture of nutrients is compressed into various dosage forms such as tablets, mini-tablets, micro-granules or micronized particles, so that they can then be coated, enveloped by a coating layer which dissolves, disintegrates or fragments in a medium with a certain pH, i.e., to allow the release of its contents, the mixture of macro-nutrients with or without micronutrients and/or bile salts, on a pH-dependent or pH-specific basis. In this system, whatever the enveloping or covering material, polymer, product or substance for involving or encapsulating or micro-encapsulating solid particles in macro or micrometric dimensions, the same shall dissolve, disintegrate or fragment within a range of Ph from 6.0 to 7.0, to promote the release of its contents in the ileum, distal small intestine and/or proximal colon, therein stimulating the secretion of intestinal hormones on amplified or supra-physiological basis, particularly PYY and GLP-1, which at high concentrations in blood produces strong inhibition of appetite, synergistically. PYY also increases energy expenditure, a significant therapeutic effect for anti-obesity, and also by the action called incretinic the GLP1 will stimulate insulin production and release concomitantly with inhibition of glucagon for an effective Type 2 anti-diabetes mellitus therapeutic effect.

### BRIEF DESCRIPTION OF THE DRAWINGS

FIG. 1 shows graphical representations of the secretion of hormones;

FIG. 2 is a flowchart of currently practiced primary surgery techniques;

FIG. 3 shows images of currently practiced primary surgery techniques;

FIG. 4 is is a schematic representation of the natural digestive transit; and

FIG. 5 is a schematic representation of the path of the pharmaceutical product of the present invention,

### CURRENT STATE OF SCIENCE

As you know, obesity is associated with various diseases accompanying it, its co-morbidities, such as type 2 diabetes mellitus, hypertension, dyslipidemia, Obstructive Sleep Apnea, steatohepatitis, and an increased risk by 3 to 5 times for various types of cancers, in addition to several other diseases that cause severe consequences, such as death rate 7 to 10 times higher, at any age, when compared to populations of normal weight, and a drastic loss in quality of life and self-esteem.

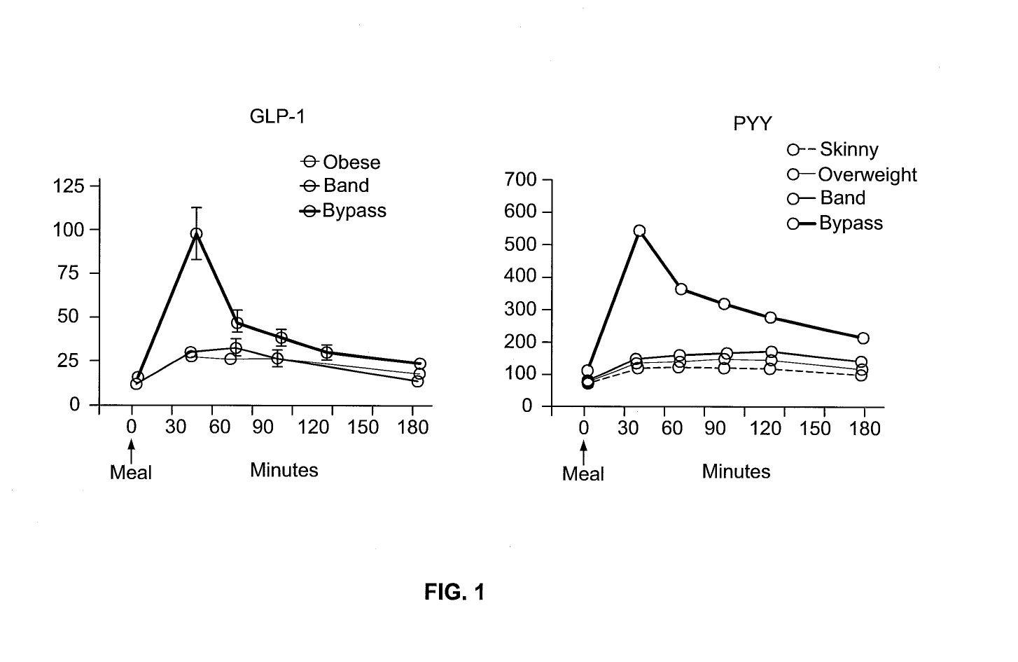

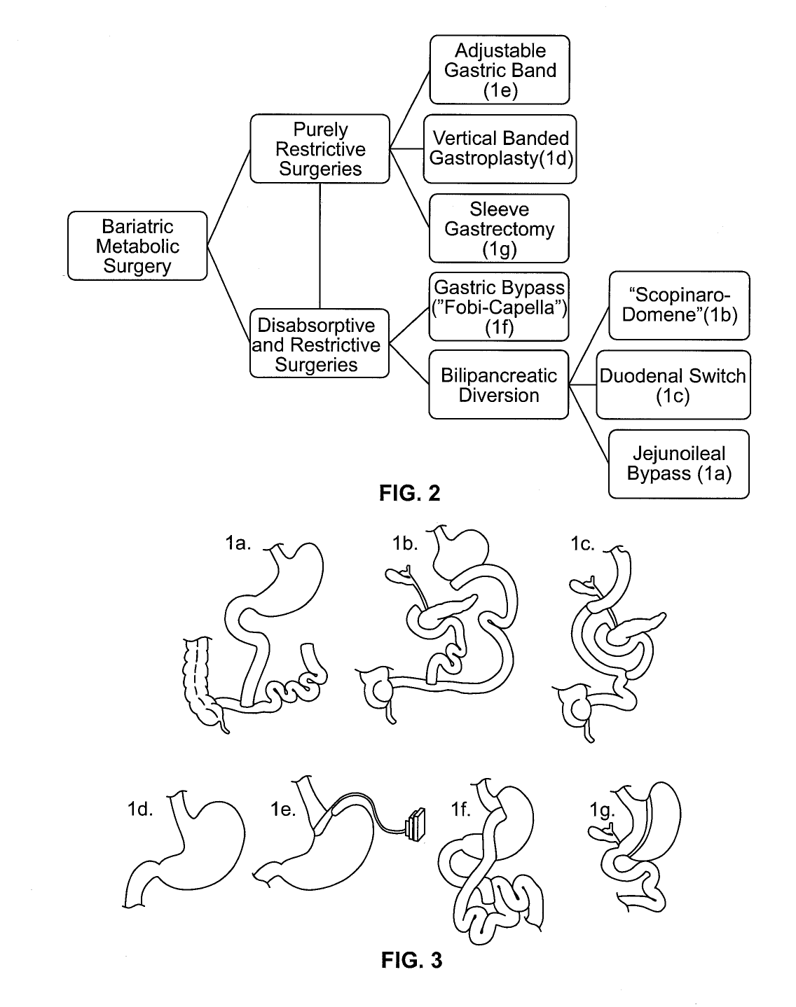

Surgeries termed Metabolic and Bariatric are, to date, the only effective way to solve obesity in its more severe degrees, as well as several serious diseases accompanying it, as above described. In surgery, such as the Roux-en-Y Gastric Bypass, in our field also known as Fobi-Capella surgery or generically known as Billiary Pancreatic Diversion, among which stand out the Scopinaro surgery and the Duodenal-Switch, there are intestinal diversions, which ultimately will cause undigested or only partially digested food to reach the distal portions of the small intestine, called ileum, and eventually up to the proximal large intestine. This unusual or non-physiological fact will cause an exacerbated response to release gut hormones, such as GLP-1 and PYY (FIG. 1). The actions of these hormones are responsible, in great part, by the control of obesity in the long term, observed in up to 80% of morbidly obese patients undergoing these surgeries. Moreover, the GLP1 with its specific incretinic action stimulates the production and release of insulin from beta cells in the pancreas, concomitantly with the inhibition of glucagon release. These actions have a powerful Type 2 Anti-diabetes effect, evidenced by early glycemic control without drugs, which is observed in 90-95% of diabetic obese patients subjected to surgery. In FIG. 1, two graphs show the secretion of these two hormones, PYY and GLP-1, after stimulation produced by a standard meal in non-operated obese patients, in skinny controls, and in patients undergoing two separate surgeries; in one of them, the Adjustable Gastric Band, there is no intestinal bypass, and the other one, the Roux-en-Y Gastric Bypass, there is diversion. It is observed clearly in the graphs the release of the two hormones is significantly higher in the Roux-en-Y Gastric Bypass than in the other groups and an intense secretion peak occurs 30 minutes after the meal. The responsible for this exacerbated secretion of these hormones is the early arrival of food components, macro-nutrients, at undigested or only partially digested state in the ileum. The distal intestine interprets this as “overeating or too fast ingestion of precious food”, what would be exceeding the digestive and/or absorptive capacity in that organism. Intestinal endocrine cells called L Cells, at higher number in the Ileum and Colon, respond to the presence of macro-nutrients by secreting supra-physiological amounts of these two hormones and maybe others, such as Oxyntomodulin. In response, these hormones break these supposed improper actions, either by decreasing appetite or slowing gastric emptying. Both PYY and GLP1 share an anorexigenic effect when they reach via blood the arcuate nucleus of the hypothalamus (CNS). PYY also stimulates energy expenditure. GLP-1, besides to incretinic action described above, reduces apoptosis of beta cells and can stimulate proliferation of these cells, resulting in hyperplasia. These actions are proven various scientific studies. Despite these highly beneficial effects, these invasive surgical procedures carry the inherent risk of mortality, early and late complications, and adverse reactions. There is also a high cost for performing these complex surgical procedures and this makes it virtually impossible to reach and treat all potential candidates because of the astronomical number of obese and/or type 2 diabetes patients worldwide. As illustrated in FIG. 2, and by images in FIG. 3, there are two groups of primary surgical techniques currently practiced, 1: purely restrictive techniques, which ultimately reduces the size of the stomach, creating functional neo-stomachs with volumes ranging from 20 to 120 ml on average, contrasting with intact stomach capacity which ranges from 1,500 to 2,000 ml. The objective is quantitatively restrict food intake, because now smaller amounts of food will be needed to fill these new tiny stomach, stretching its muscular wall to the maximum physiological limit, which in turns stimulate receptors sensitive to stretching and distortion and generate electric signals. These electrical signals, conducted by afferent branches of the vagus nerve, reach the satiety center in the hypothalamus and this induces a physiological response called satiation, which means stopping the act of eating, as well as satiety, which means loss of interest in food for a specific period of hours; 2: disabsorptive-restrictive techniques that include intestinal diversions, which promote the early arrival of partially digested food in the distal segments of the small intestine, such as in the ileum. This fact, unnatural, stimulates the exacerbated or supra-physiological secretion of gut hormones, particularly PYY (peptide YY) and GLP-1 (glucagon-like polypeptide 1), which by their anorexigenic actions, shared by both hormones, and the incretinic action of GLP-1 contribute significantly to the success of these surgeries, both by inducing weight loss and by controlling type 2 diabetes mellitus. In the first group, of purely restrictive surgeries, as shown in the flowchart in FIG. 2, the following techniques are applied: Adjustable Gastric Band (AGB) (1e); Vertical Banded Gastroplasty (VBG) (1d); and the latest of them, the Vertical or Sleeve Gastrectomy (SG) (1g). The surgeries referred to in this group do not have the benefit of stimulated release of these hormones, since the transit of food is natural and the digestive process suffers no interference. In Sleeve Gastrectomy, there is some evidence that due to accelerated gastric emptying there is an increased release of GLP-1 and PYY, but less than in Roux-en-Y Gastric Bypass. For this reason, these surgeries are less effective both for weight loss and for controlling diabetes.

In the case of the second group, of disabsorptive-restrictive surgeries, the techniques employed are divided into two subgroups, namely: Gastric Bypass or “Fobi-Capella” (1f), which is the most frequently performed in Brazil and around the world; and the Bilio-Pancreatic Diversion, such as “Scopinaro” (1b), Duodenal Switch (1c), and Jejuno-ileal Bypass (1a). The latter is no longer performed.

In the second group, in general, the food leaving the stomach, not yet digested, passes through the proximal small intestine (duodenum and jejunum) and is surgically diverted to the distal portions thereof (distal jejunum and ileum), and this stimulates the release of the hormones referred to above and even more intensively in Bilio-Pancreatic Diversion surgeries because the undigested or partially digested food directly reaches the ileum, the most distal portion of the small intestine, where the number of L cells producing these hormones is maximal, releasing them in even greater quantities.

As an alternative to the practice of those surgery described above, the pharmaceutical industry has produces molecules similar to PYY and GLP-1 hormones, aiming to produce, pharmacologically, its potentially beneficial effects. However, these molecules transformed into drugs have the disadvantages of high cost, the need for continuous use via parenteral route, significant adverse reactions, and the induction of drug resistance.

### PURPOSE/SCOPE OF THE INVENTION

The pharmaceutical product in focus, subject of this application, in different possible dosage forms such as tablets or mini-tablets or micro-granules or micronized particles, coated/enveloped by polymers or product for encapsulation or any substance that serves as envelope/cover/wrap, including those suitable for micro-encapsulation, which are dissolved, disintegrated or destroyed or fragmented by the influence of the medium pH or intestinal contents where they are, that is, on a pH-dependent or pH-specific basis, was developed with the particular purpose of releasing their contents, a mixture of macro-nutrients with or without the presence of micro-nutrients and/or bile salts, in the ileum and/or proximal colon. After being released, the mixture promotes an exacerbated/amplified stimulus in L Cells, at highest number in these referred intestinal segments, to release increased amounts, ranging from 3 to 10 times more than normal, the hormones GLP-1 and PYY and other hormones eventually, such as, for example, Oxyntomodulin. All because of this new non-invasive method.

By means of this product in question, therapeutic effects, such as anti-obesity, type 2 anti-diabetes, and other metabolic benefits can be obtained. These effects are induced by the inhibitory actions on appetite, shared by the two hormones, and the stimulation of energy expenditure by PYY plus the incretinic action of GLP-1, similarly to what is obtained by bariatric and metabolic surgeries described above and by the same mechanisms that produce the release of these hormones, but avoiding their adverse reactions, early and late complications and eventually mortality, all inherent to an invasive complex procedure that changes the gastrointestinal anatomy and physiology.

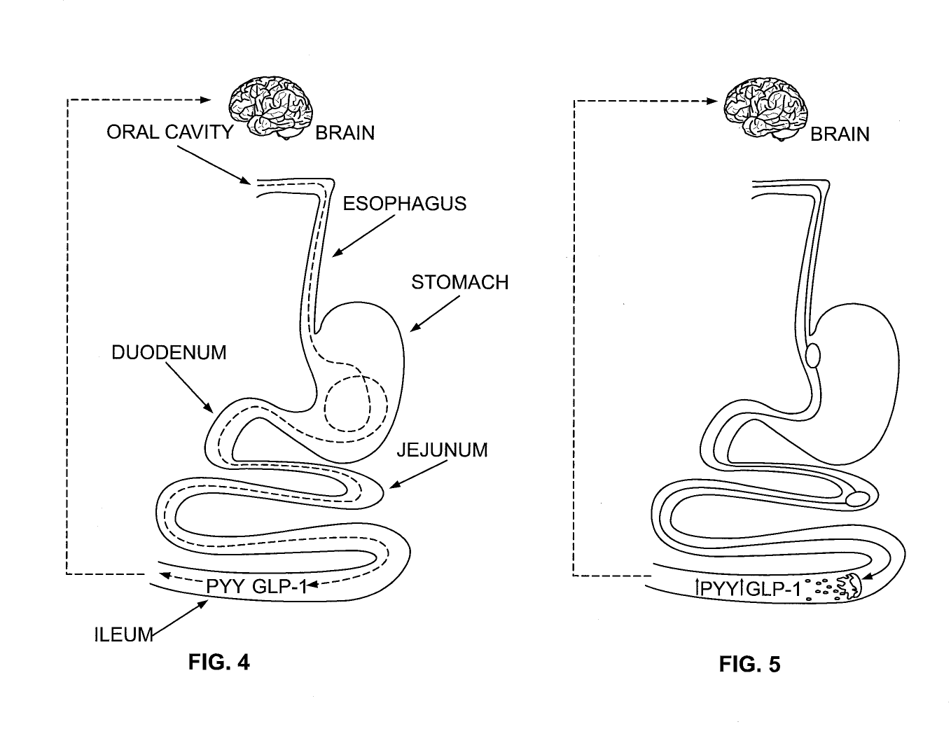

Once superficially explained, the product is now described in detail, by means of the attached drawings, from FIG. 4: FIG. 4—Schematically shows all natural and physiological digestive transit, without the intake of the pharmaceutical product, of an individual not subject to any type of bariatric and metabolic surgery. As shown, when the intake of food occurs in the oral cavity, it is already being digested by the action of some salivary enzymes, but not in significant extent. The oral pH is about 7.0. In the esophagus the standard pH is also 7.0, and this organ no digestive action takes place. In the stomach, where pH is very acidic (0.9 to 2.0), gastric juice acts by activating the proteolytic enzyme pepsin, which is responsible for partial hydrolysis of certain protein molecules. In the duodenum, proximal portion of the small intestine, the content leaves the stomach at a very acid pH and comes into contact with bile salts, pH between 8.0 to 8.5, and the pancreatic juice, pH between 8.5 and 9.0. This mixture of digestive juices modifies the duodenum pH to a pH between 5.0 and 5.5. Throughout the jejunum the pH becomes gradually less acidic and when it reaches the ileum (the distal small intestine) the pH ranges from 6.0 to 6.5. Upon arriving at the distal jejunum and Ileum, virtually all macro-nutrients have been digested and absorbed. Therefore, it is not physiological macromolecules (proteins, lipids, carbohydrates) reaching the Ileum still undigested. Such abnormal fact, if it occurs, will stimulate the release of large amounts of PYY and GLP-1 hormones, which reach the arcuate nucleus in the hypothalamus and produce marked inhibition of appetite and stimulate energy expenditure. GLP-1, in turn, stimulates beta cells to produce and secrete more insulin when it reaches the pancreas.

FIG. 5—Schematically shows the path of the pharmaceutical product, subject of this patent application.

In accordance with the attached drawings, the pharmaceutical product to induce exacerbated endogenous release of gut hormones, pyy and glp1, with therapeutic effect on obesity and type 2 diabetes mellitus, object of this patent application, is coated with polymeric layer of Ph-dependent disintegration and releases its contents in Ileum/Colon, creating a system to stimulate the release of gut hormones PYY and GLP-1 and to obtain a therapeutic effect on obesity in type 2 DM, comprising an associative formula of macro-nutrients, such as proteins, peptones, polypeptides, lipids (fats and oils), long-, medium- or short-chain triglycerides, saturated or unsaturated fatty acids, poly- and oligosaccharides, bile salts, in addition to micronutrients, such as vitamins and minerals, in any possible composition proven effective for this purpose.

Such formula can be prepared in the form of tablets or pills, mini-tablets, micro-granules or micronized particles, receiving a coating or envelope or wrap of polymers or products or substances for wrapping/coating, including those deemed suitable for micro-encapsulation, for example, the polymer “Eudragit”; whatever the coating, it will have to undergo pH-dependent disintegration or dissolution or fragmentation in the intestinal lumen, i.e., at a specific pH of a given intestinal region and for the purpose of this invention, whatever the Ileum and/or proximal colon where the pH ranges from 6.0 to 7.0, and thus obtaining the pharmaceutical product in question.

As shown in FIG. 5, when ingested, the product thus obtained travels through the esophagus up to the stomach where, despite the extremely acidic pH, the coating or wrap, according to the design in question, does not suffer any change in its molecular structure, remaining intact and going through duodenum, jejunum, and reaching the ileum. According to the design, upon reaching the distal small intestine (ileum), the coating suffers disintegration by the action of the target pH found at this site (ileum), between 6.0 and 6.5, releasing in ileum lumen the mixture of macro-nutrients, with or without micro-nutrients and bile salts, contained in the pharmaceutical product. This stimulates the secretion of PYY and GLP-1 hormones to achieve abnormal levels, 3 and 10 times higher, i.e., supra-physiological levels for these hormones, which are produced by L cells present in higher number exactly in this region of the small intestine, i.e., in the distal portion called ileum. This induced effect causes loss of appetite and satiety in patients, even before eating.

Furthermore, by incretinic action, that is, the action stimulating insulin secretion, and by inhibitory action of glucagon secretion (hyperglycemic hormone) of GLP-1, a better glycemic control is achieved in diabetic patients.

Thus, by noninvasive and of access method, that produced and performed in bariatric and metabolic surgery is mimicked without the inherent risks, such as eventual mortality, early and late complications and/or undesired adverse reactions, and, at the same time, facilitated and made possible the access to billions of people suffering from obesity and/or type 2 diabetes.

1) The pharmaceutical product to induce endogenous exacerbated release of gut hormones, pyy and glp1, with therapeutic effect on obesity and type 2 diabetes mellitus, consisting of associative formula of macro-nutrients, such as proteins, peptones, polypeptides, lipids (fats or oils), long- and/or medium- and/or short-chain triglycerides, cholesterol, saturated or unsaturated fatty acids, poly- and oligosaccharides, bile salts, in addition to micronutrients, such as vitamins and minerals, in any combination of these substances which proves to be effective for purpose of the invention, said formula being prepared in any possible dosage form, whatever tablets or pills or mini-tablets or micro-granules or micronized particles, receiving coating or envelop or wrap by polymers or products or substances for coating, such as “Eudragit”, for example, or any other product or substance for coating any of the dosage forms types alluded above, including encapsulated products found to be suitable for micro-encapsulation, thus obtaining the pharmaceutical product in question, which, thus made, after ingested travels intact through the esophagus up to the stomach, following through the duodenum, jejunum, and reaches the ileum, wherein said pharmaceutical product, only upon reaching the distal small intestine, the ileum, and/or eventually the proximal colon, has its coating disintegrated, dissolved or fragmented by the action of intraluminal pH as found in the ileum and/or proximal colon with a pH value between 6.0 and 7.0, thus releasing in the Ileum and/or proximal colon lumen the macro nutrients, with or without micro-nutrients, with or without bile salts, with or without other substances described above, contained in the pharmaceutical product, to stimulate or incite the secretion of anorexigenic hormones (PYY and GLP-1) and incretinic hormone (GLP-1) by L cells, when this cells comes in contact with undigested or partially digested macronutrients, and thus produces a major or supra-physiological stimulus for these L cells, and that these two hormones mentioned, released at high levels, well above normal, will cause potentially therapeutic effects for obesity, in any degree, and also for type 2 diabetes mellitus, by producing strong inhibition of appetite or loss of appetite and satiety in the patient, even before eating, in addition to, by stimulating the release of insulin and inhibiting glucagon, the hyperglycemic hormone, produce better glycemic control in diabetic patients.
